(19) United States
(12) Patent Application Publication
(10) Pub. No.: US 2019/0270957 A1
(43) Pub. Date: Sep. 5, 2019

(54) Anaerobic Fermentation Apparatus

(71) Applicant: Tze-Ming Kao, Taipei (TW)

(72) Inventor: Tze-Ming Kao, Taipei (TW)

(21) Appl. No.: 15/908,923

(22) Filed: Mar. 1, 2018

(51) Int. Cl. C12M 1/00; B65D 88/12

(52) U.S. Cl. CPC C12M 29/06; C12M 41/44; B65D 88/128; C12M 29/24

(57) ABSTRACT

An anaerobic fermentation apparatus has at least one fermentation tank. Each fermentation tank has a container, a pumping pipe, a conveying pipe, and a spray pipe. The pumping pipe communicates with the top of the container. The conveying pipe is mounted in the container. The spray pipe is mounted in the container and has a trunk and multiple branches. The trunk communicates with the conveying pipe. Each branch communicates with the trunk and has two nozzles formed through the wall of the branch. An imaginary connection line that connects the two nozzles is oblique with respect to the axis of the branch as viewed from the top, causing multiple vortexes at the bottom of the container to make the organic substances stirred thoroughly and the fermentation processing more evenly, and increase the efficiency of the fermentation.

### BACKGROUND OF THE INVENTION

### 1. Field of the Invention

The present invention relates to an anaerobic digestion processing device, especially to an anaerobic digestion processing device for the organic substances of anaerobic fermentation.

### 2. Description of the Prior Arts

Marsh gas is a kind of flammable gas generated by anaerobic bacteria fermentation. The main compositions of marsh gas include methane, carbon dioxide and hydrogen sulfide. The sources of marsh gas include manure, sewage, garbage, and other biodegradable organic substances. While marsh gas generated by abandoned organic substances is used as fuel to generate power, marsh gas can also be utilized to clean the environment and deal with the garbage at the same time. Thus, development in the applications of marsh gas has been valued gradually. The conventional fermentation apparatus has an air tight container. Organic substances and the anaerobic bacteria culture medium are fed together into the container and then the container is sealed to be isolated from the outer air. In the anoxic environment, the anaerobic bacteria start to grow and propagate and cause fermentation of the organic substances.

However, the conventional fermentation apparatus only accumulates the organic substances in the container, so the anaerobic bacteria will start to propagate at the bottom of the container where less oxygen is contained, and then the anaerobic bacteria grow gradually upwards from the bottom to the top. Therefore, the fermentation is slow and has no uniformity. Thus, an anaerobic fermentation apparatus disclosed in Taiwan patent No.I535669 is provided. With reference to FIG. 11, the main objective of the abovementioned patented anaerobic fermentation apparatus 90 is to mount a spray pipe 92 at the bottom of the container 91, and then set a pumping pipe 93 at the upper apex of the container 91, and use a circulation unit 94 to make the marsh gas, which is at the top of the container 91, circulate from the pumping pipe 93 to the spray pipe 92, and then eject the marsh gas by a nozzle 96 of the spray pipe 92 to stir the organic substances 95 in order to make the fermentation process faster and more evenly.

However, as the structure of the spray pipe 92 is a single elongated straight pipe, with reference to FIGS. 11 and 12, the stir range of the ejection is limited. Moreover, the nozzle 96 only ejects directly upward, so the effect of the stirring is not as ideal as expected. In summary, the fermentation efficiency of the aforementioned anaerobic fermentation apparatus 90 needs to be improved.

To overcome the shortcomings, the present invention provides an anaerobic fermentation apparatus to mitigate or obviate the aforementioned problems.

### SUMMARY OF THE INVENTION

The main objective of the present invention is to provide an anaerobic fermentation apparatus that can generate multiple vortexes inside a container with a spray pipe and a nozzle group, thereby making the fermentation processed faster and more evenly.

The anaerobic fermentation apparatus comprises a circulation unit and at least one fermentation tank. The circulation unit comprises a pump and at least one storage tank. The pump and the at least one storage tank communicate with each other. Each one of the at least one fermentation tank has a container, a pumping pipe, a conveying pipe, a valve, and a spray pipe. The container has at least one opening. The pumping pipe communicates with a top of the container and the pump. The conveying pipe communicates with the at least one storage tank and is mounted in one of the at least one opening of the container. The conveying pipe extends in the container. The valve is mounted on the conveying pipe and is located outside the container. The spray pipe is mounted in the container and has a trunk and multiple branches. The trunk communicates with an end of the conveying pipe which is extending in the container. The multiple branches are round tubes. Each one of the multiple branches communicates with the trunk and has at least one nozzle group. Each one of the at least one nozzle group has two nozzles. The two nozzles are formed through tube walls of the branches. An imaginary connection line that connects the two nozzles is oblique with respect to an axis of the branches as viewed from the top.

The present invention changes the structure of the spray pipe and adds multiple branches in order to make the conventional single elongated straight pipe extend transversely by the branches so that it can enlarge the range within which the cycling marsh gas is stirred after the ejection. Besides, the connection line that connects the two nozzles of the nozzle group is oblique with respect to the axis of the branches, and the branches are round tubes, so the directions of the ejection of the two nozzles are non-parallel. Therefore, two ejections at different angles and in different directions cause multiple vortexes at the bottom of the container, and the vortexes make the organic substances stirred thoroughly, and then make the fermentation processed more evenly and increase the efficiency of the fermentation.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of an anaerobic fermentation apparatus in accordance with the present invention;

FIG. 2 is a top view of the anaerobic fermentation apparatus in FIG. 1;

FIG. 3 is a partial enlarged top view of the anaerobic fermentation apparatus in FIG. 1, shown with a trunk and a branch;

FIG. 4 is a front cross-sectional view of the anaerobic fermentation apparatus in FIG. 1, shown with the branch;

FIG. 5 is a side cross-sectional view of a first embodiment of anaerobic fermentation apparatus in FIG. 1, shown filled with organic substances;

FIG. 6 is a side cross-sectional view of the first embodiment of the anaerobic fermentation apparatus in FIG. 1, shown with the cycling marsh gas;

FIG. 7 is a side cross-sectional view of the first embodiment of the anaerobic fermentation apparatus in FIG. 1, shown draining the water produced from the organic substances;

FIG. 8 is a top view of another embodiment of an anaerobic fermentation apparatus in accordance with the present invention, shown with multiple fermentation tanks;

FIG. 9 is a top view of still another embodiment of an anaerobic fermentation apparatus in accordance with the present invention, shown with the ejecting pipe;

FIG. 10 is a side cross-sectional view of a second embodiment of the anaerobic fermentation apparatus in accordance with the present invention, shown with the container;

FIG. 11 is a side cross-sectional view of the anaerobic digestion processing device in accordance with the prior art, shown the cycling marsh gas; and

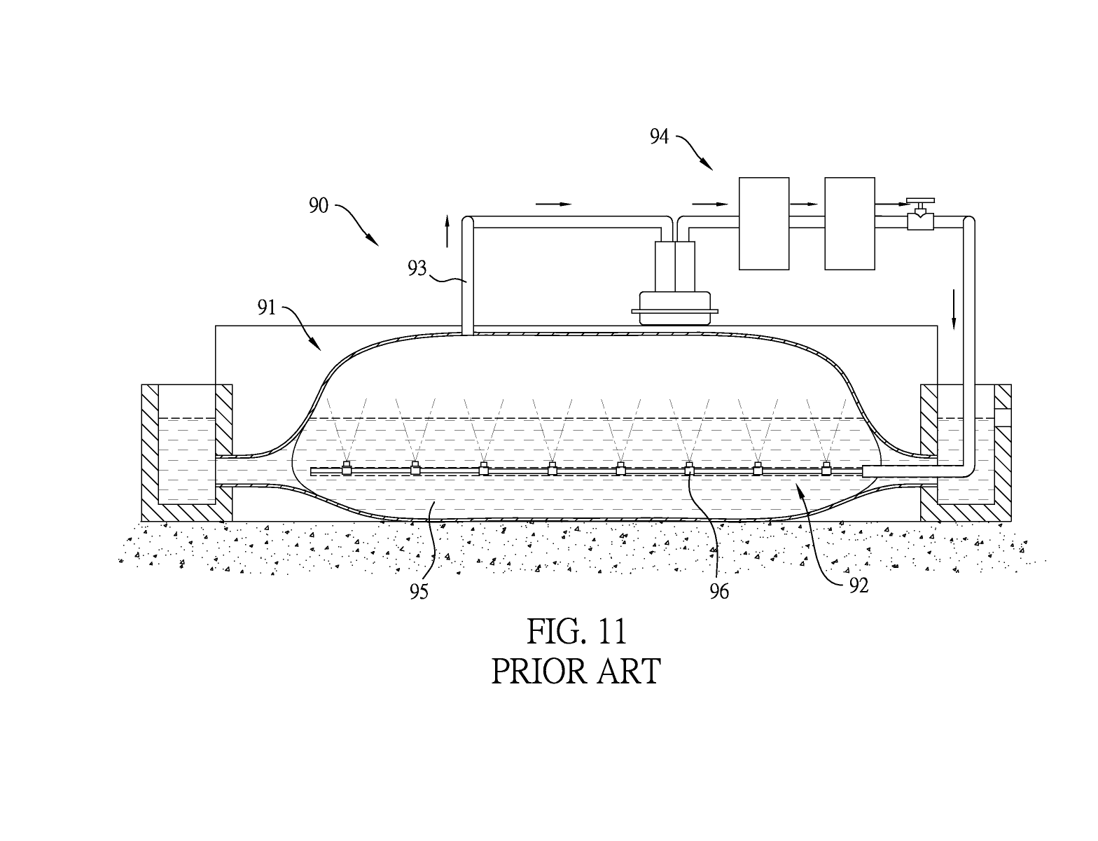

FIG. 12 is a top view of the anaerobic digestion processing device in FIG. 11.

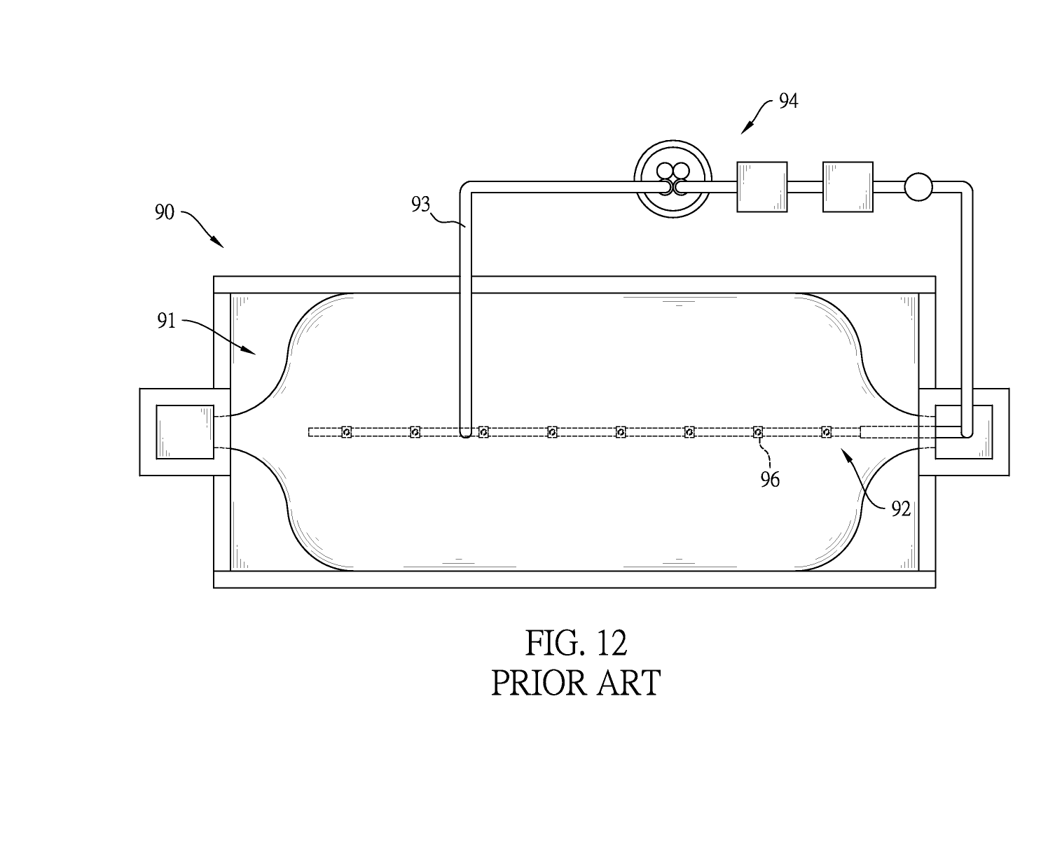

### DETAILED DESCRIPTION OF THE PREFERRED EMBODIMENTS

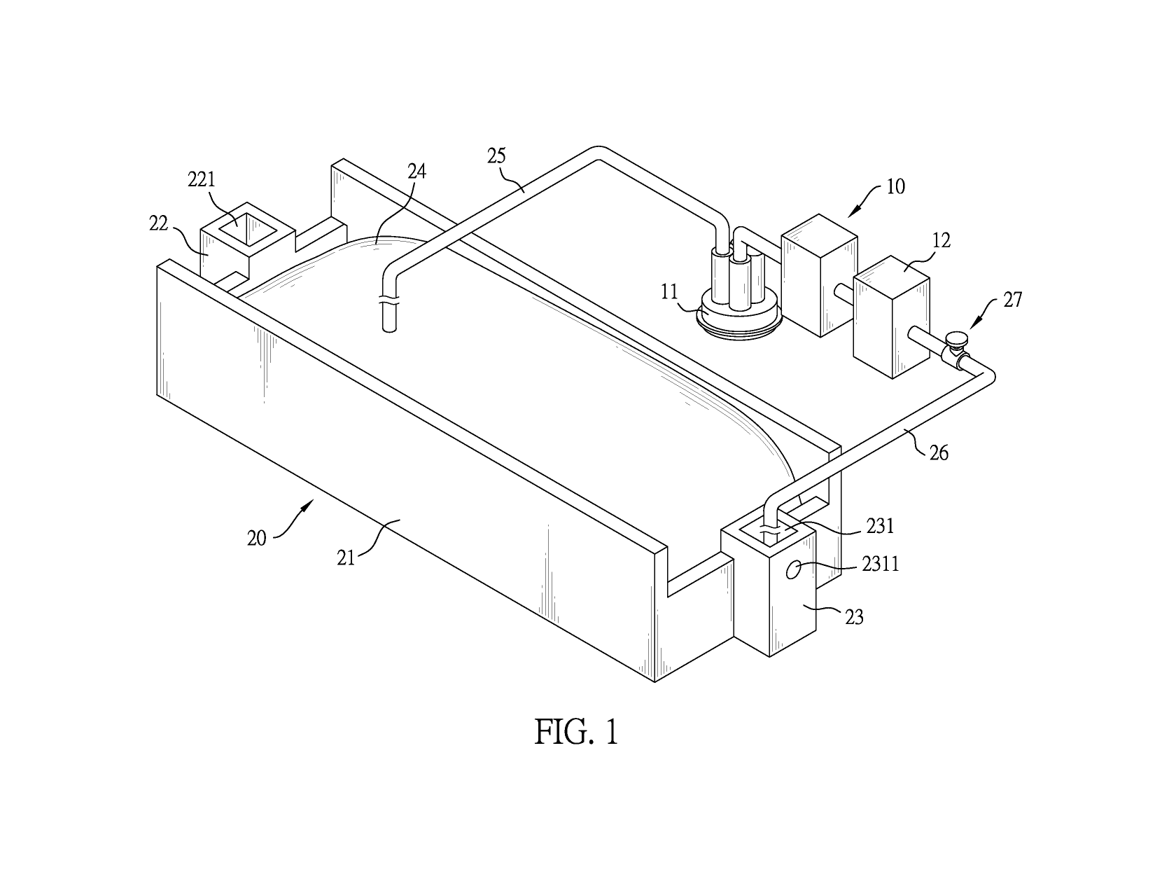

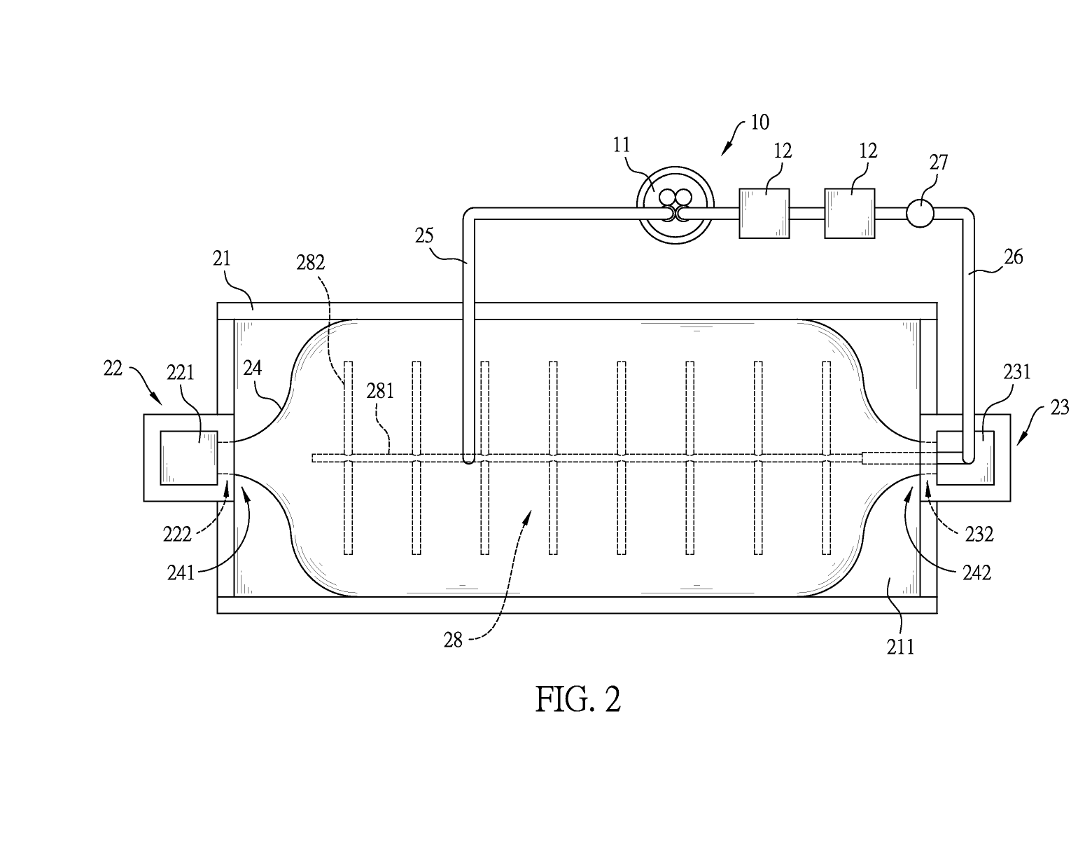

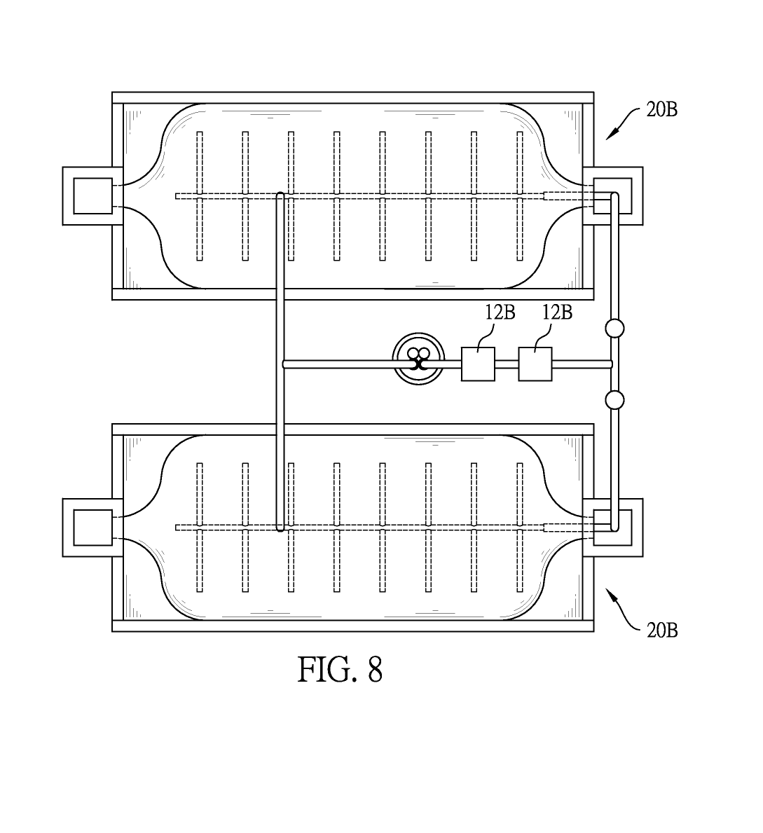

With reference to FIGS. 1 and 2, an anaerobic fermentation apparatus in accordance with the present invention comprises a circulation unit 10 and at least one fermentation tank 20. The circulation unit 10 has a pump 11 and at least one storage tank 12 which are communicating with each other. The at least one storage tank 12 also communicates with the at least one fermentation tank 20. Specifically, the amount of the at least one storage tank 12 in the first embodiment of the present invention is, but not limited to, two. In the first embodiment, the two storage tanks 12 communicate with each other at first and then communicate with the pump 11. But in another embodiment, the multiple storage tanks 12 can also communicate with the pump 11 respectively but do not communicate with each other. Beside, the first embodiment of the present invention only has one fermentation tank 20, but there can be also two fermentation tanks 20B in another embodiment as shown in FIG. 8. In this way, the two storage tanks 12B communicate with each other at first and then communicate with the two fermentation tanks 20B. But the two storage tanks 12 can also communicate with the pump 11 respectively and then communicate with the two fermentation tanks 20B respectively.

With reference to FIGS. 1, 2, and 5, in the first embodiment, each one of the at least one fermentation tank 20 has a containment barrier 21, a first transporting base 22, a second transporting base 23, a container 24, a pumping pipe 25, a conveying pipe 26, a valve 27, and a spray pipe 28.

The containment barrier 21 surrounds a receiving space 211. The first transporting base 22 is mounted on an outer wall of the containment barrier 21 and has a first gateway 221 and a first connecting opening 222. The first gateway 221 is formed in the first transporting base 22. That is, the first transporting base 22 surrounds the first gateway 221. The first connecting opening 222 is formed through a wall of the containment barrier 21 and communicates with the first gateway 221 and the receiving space 211. The second transporting base 23 is mounted on the outer wall of the containment barrier 21 opposite to the first transporting base 22 and has a second gateway 231 and a second connecting opening 232. The second gateway 231 is formed in the second transporting base 23. That is, the second transporting base 23 surrounds the second gateway 231. The second connecting opening 232 is formed through the wall of the containment barrier 21 opposite to the first connecting opening 222 and communicates with the second gateway 231 and the receiving space 211. Besides, the second transporting base 23 has an outlet 2311 formed through the second transporting base 23 and communicating with the second gateway 231. In a preferred embodiment, the outlet 2311 is used to drain water produced from the organic substances to the exterior space of the fermentation tank 20, but the fermentation tank 20 can be implemented without the outlet 2311, and the water produced from the organic substances will be drained from the second gateway 231.

The container 24 is mounted in the receiving space 211, and has at least one opening. In a preferred embodiment, the container 24 has a first opening 241 and a second opening 242. The first opening 241 is formed through the container 24, faces the first transporting base 22, and communicates with the first gateway 221 of the first transporting base 22 via the first connecting opening 222. That is, objects can enter into the inner space of the container 24 by entering the first gateway 221 of the containment barrier 21 and passing through the first connecting opening 222. The second opening 242 is formed through the container 24, faces the second transporting base 23, and communicates with the second gateway 231 of the second transporting base 23 via the second connecting opening 232. That is, objects can enter into the inner space of the container 24 by entering the second gateway 231 of the containment barrier 21 and passing through the second connecting opening 232.

The pumping pipe 25 communicates with a top of the container 24 and the pump 11. The pumping pipe 25 is used to extract the marsh gas that is at the top of the container 24 into the pump 11. The conveying pipe 26 communicates with the at least one storage tank 12 and is mounted in one of the at least one opening of the container 24, and the conveying pipe 26 extends in the container 24. Specifically, the conveying pipe 26 is mounted in the second gateway 231 of the second transporting base 23, and the conveying pipe 26 is mounted in the second opening 242 of the container 24 and extends inside the container 24. By this way the fermentation tank 20 can communicate with the circulation unit 10 and forms a cycling pipeline. Besides, the valve 27 is mounted on the conveying pipe 26 and located outside the container 24. The valve 27 is used to control opening and closing of the pipe between the storage tank 12 and the container 24. In the embodiment that has multiple fermentation tanks 20, the valve 27 in each fermentation tank 20 respectively controls the flow direction of the marsh gas in the storage tank 12 in order to achieve the effect of individual cycling.

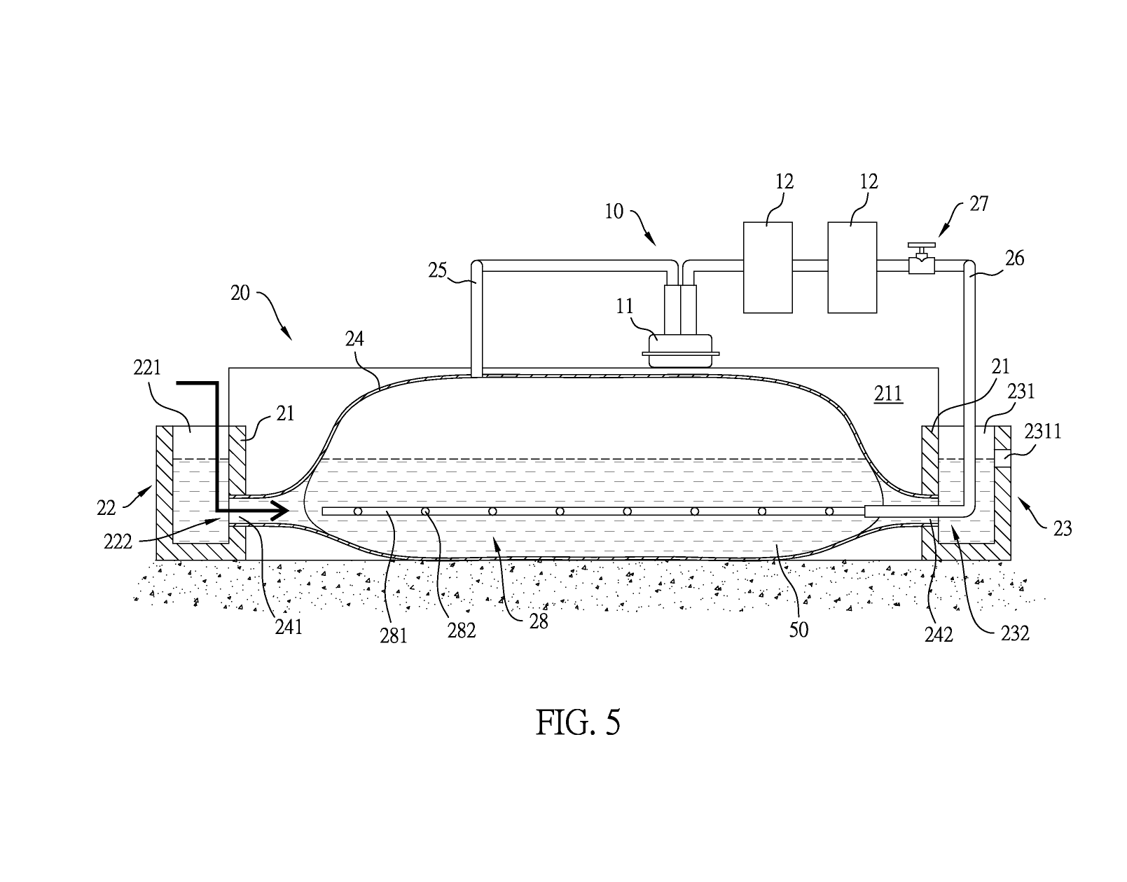

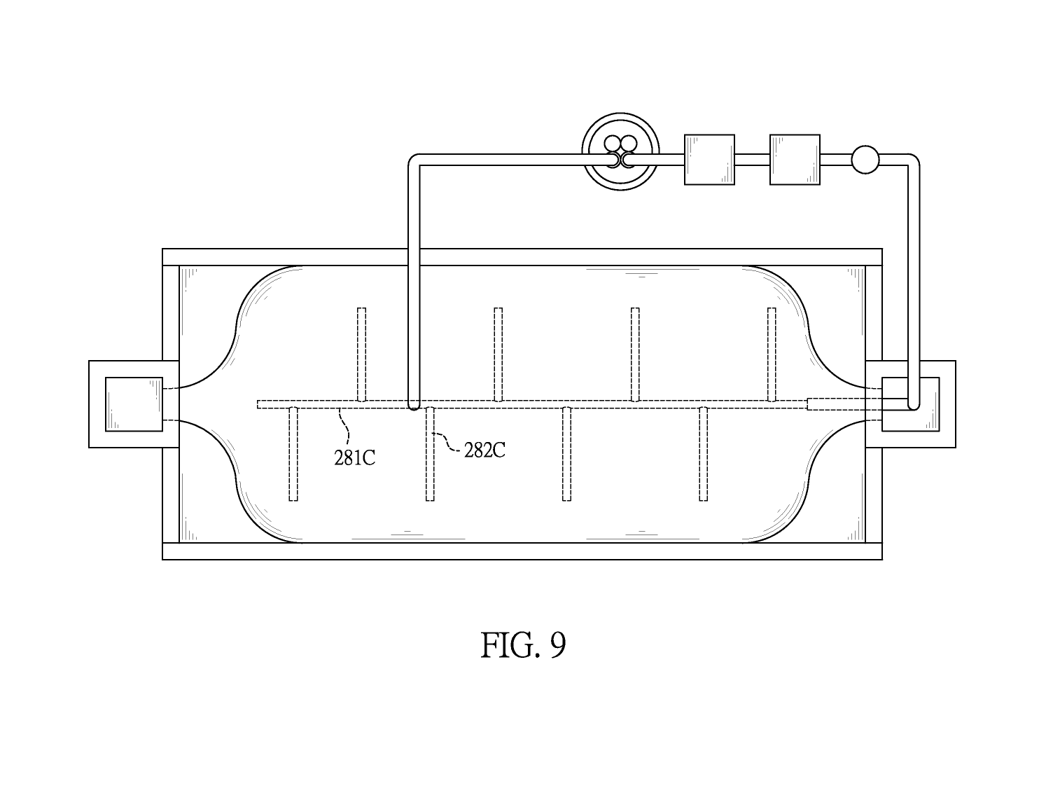

With reference to FIGS. 2 and 5, the spray pipe 28 is mounted in the container 24 and has a trunk 281 and multiple branches 282. The trunk 281 communicates with an end of the conveying pipe 26 which is extending in the container 24. The multiple branches 282 are round tubes. Each one of the multiple branches 282 communicates with the trunk 281. In a preferred embodiment, the branches 282 and the trunk 281 are disposed on a same surface. The branches 282 are divided into two groups respectively on two sides of the trunk 281, the two groups of the branches 282 correspond in position, and the branches 282 in the same group are spaced apart from each other. In other words, there are two branches 282 mounted on the both sides of the trunk 281 at the same distance to an end of the trunk 281, and the connecting point of the branches 282 and the trunk 281 forms a cross. This is a preferred embodiment of the spray pipe 28, but the structure of the spray pipe 28 is not limited to the above mentioned structure. The trunk 281 and the branches 282 can be disposed on different surfaces, and the branches 282 can also connect to the trunk 281 in any other way. For example, in still another embodiment as shown in FIG. 9, the two groups of the branches 282C are staggered with respect to each other. In other words, at the same distance to the end of the trunk 281C on the both sides of trunk 281C, only one branch 282C is connected to one side of the trunk 281C, and the trunk 281C and the branch 282C form a T shape at the connecting point. Besides, in these two preferred embodiments of the spray pipe 28, the branches 282 connect to the trunk 281 vertically, but it is not limited thereto.

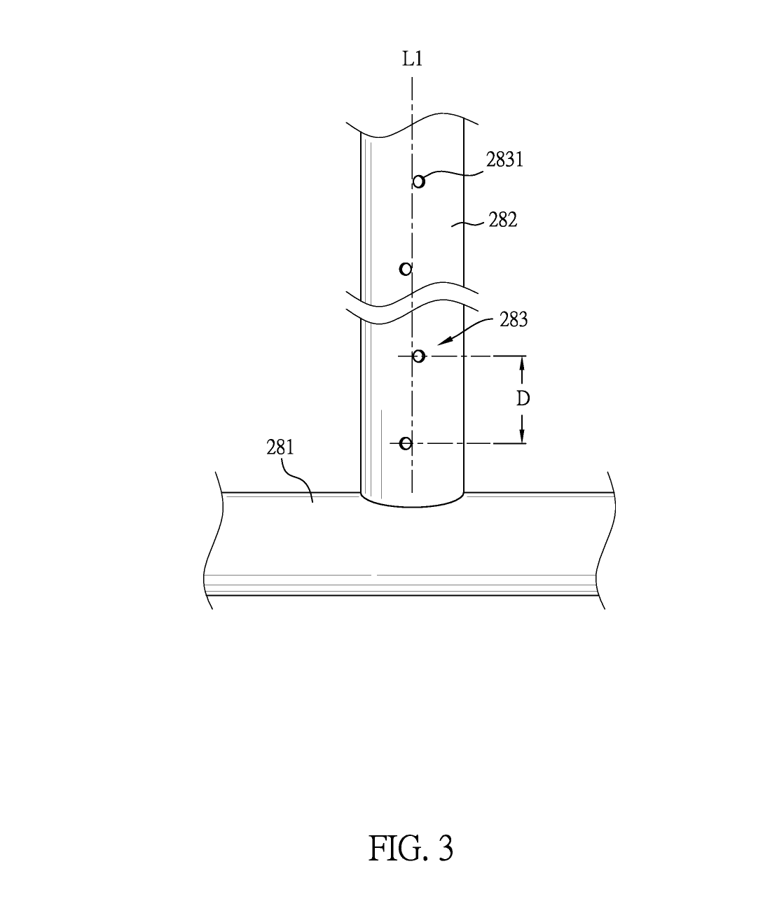

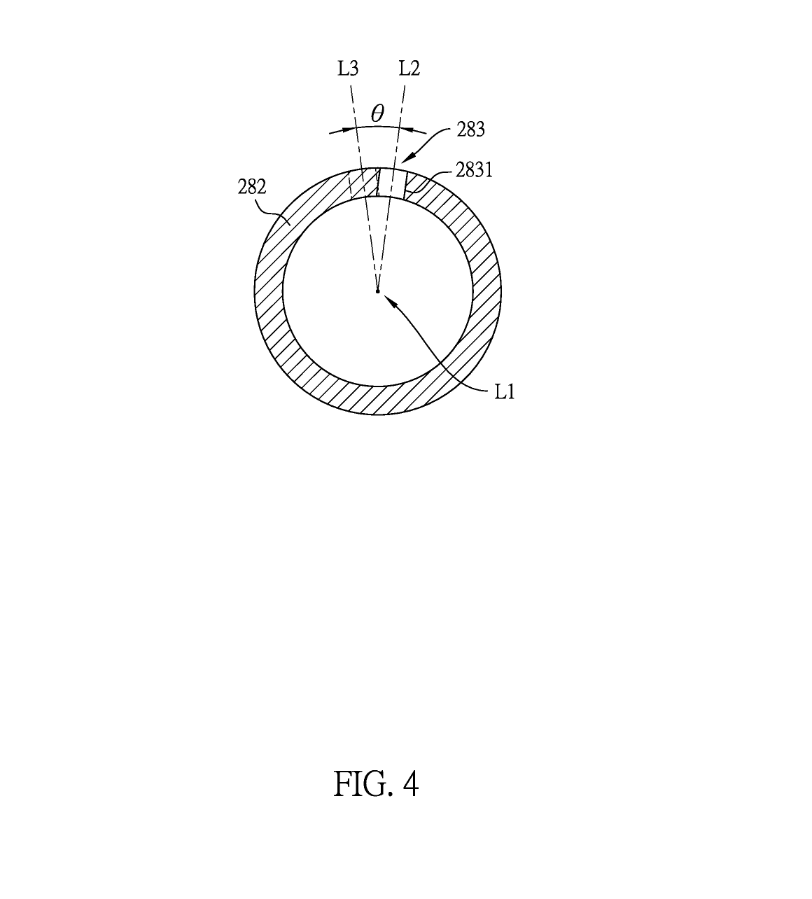

In addition, with reference to FIG. 3, each one of the multiple branches 282 has at least one nozzle group 283. In a preferred embodiment, the at least one nozzle group 283 in each one of the multiple branches 282 includes multiple nozzle groups 283, and a distance between each two adjacent ones of the nozzle groups 283 is from 20 to 40 centimeters, but the distance between each two adjacent ones of the nozzle groups 283 is not limited to the above mentioned range. Furthermore, each one of the at least one nozzle group 283 has two nozzles 2831, the two nozzles 2831 are formed through a tube wall of the branch 282, and an imaginary connection line that connects the two nozzles 2831 is oblique with respect to an axis L1 of the branch 282 as viewed from the top. That is, the two nozzles 2831 are located in different positions on the axial direction (with reference to FIG. 3), and the two nozzles 2831 are located in the different positions on the circumference of the branches 282 as viewed from the front (with reference to FIG. 4). Besides, in each one of the at least one nozzle group 283, two projections of the two nozzles 2831 on a section of the branch 282 connect to the axis L1 of the branch 282 and form two imaginary connecting lines L2, L3 respectively, and an angle between the two imaginary connecting lines L2, L3 is, but not limited to, from 10 to 15 degrees. Furthermore, in each one of the at least one nozzle group 283, a distance D between the two nozzles 2831 along the axis L1 of the branch 282 is, but not limited to, from 0.5 to 2 centimeters.

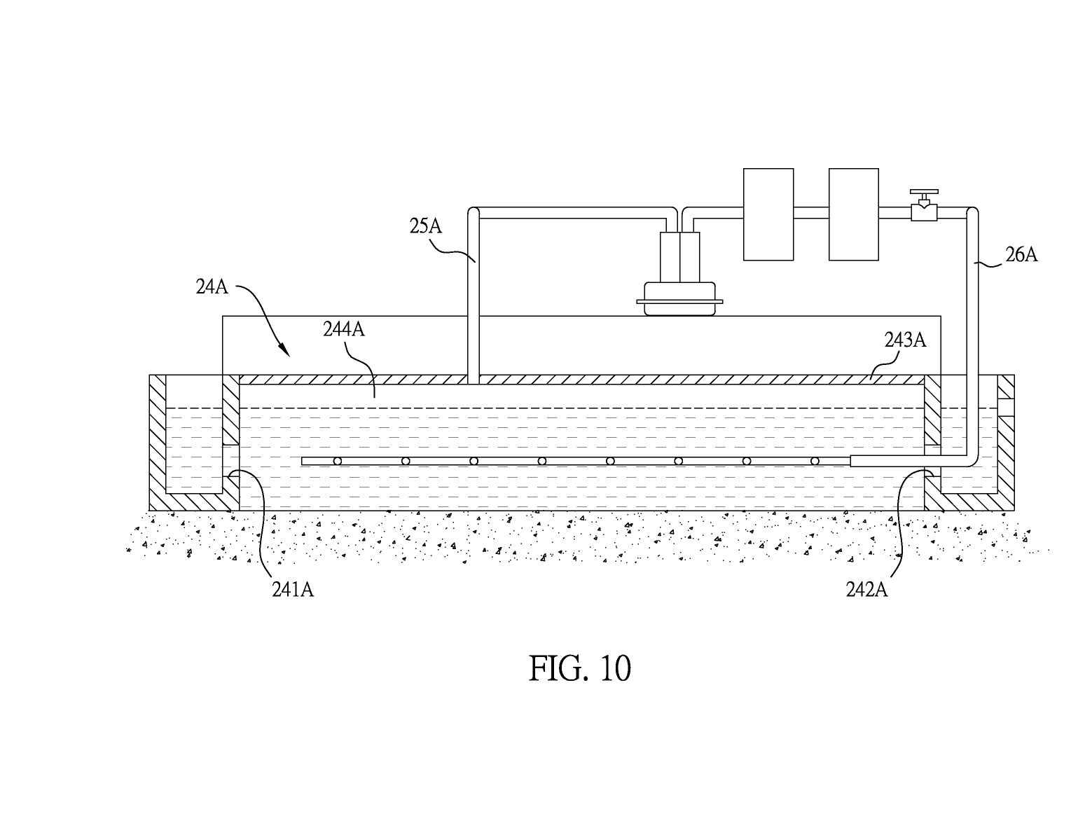

With reference to FIG. 10, in the second embodiment, the container 24A is made of cement or brick and has a first opening 241A, a second opening 242A, a cover 243A. The first opening 241A is formed through a wall of the container 24A. The second opening 242A is formed through the wall of the container 24A opposite to the first opening. The cover 243A is hermetically mounted on tops of inner walls of the container 24A and forms an inner space 244A, and the inner space 244A communicates with the first opening 241A and the second opening 242A. The pumping pipe 25A is formed through the cover 243A and communicates with the inner space 244A. The conveying pipe 26A is mounted in the second opening 242A of the container 24A and extends in the inner space 244A of the container 24A.

With reference to FIG. 5, for use, organic substances 50 such as sewage, waste of agriculture breeding waste, and kitchen waste are fed into the container 24 by passing through the first gateway 221 of the first transporting base 22 and the first opening 241 of the container 24. The organic substances 50 cover the spray pipe 28. The nutrient fluid of the anaerobic bacteria is poured into the container 24 by the first gateway 221 and the first opening 241. Because the air inside the container 24 is isolated from the outer space by the organic substances 50, the space inside the container 24 becomes an oxygen deficit environment. Thus, the anaerobic bacteria start to grow and propagate and cause fermentation of the organic substance 50.

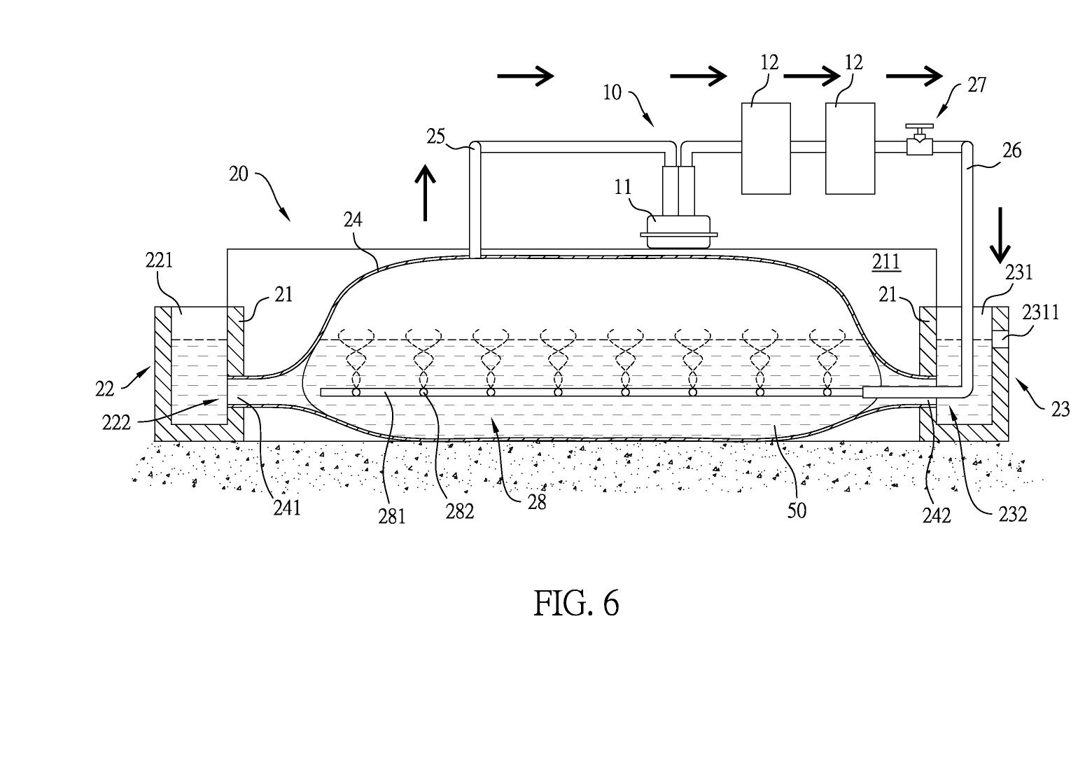

With reference to FIG. 6, during the fermentation process, the organic substances 50 start to be decomposed and to generate marsh gas, marsh gas slurry, and organic fertilizers. The marsh gas floats at the top of the container 24, and the marsh gas slurry and the organic fertilizers are deposited at the bottom of the container 24. When the marsh gas has been generated, the pump 11 of the circulation unit 10 starts to work and extract the marsh gas from the container 24 by the pumping pipe 25. The marsh gas passes through the pump 11 and then is stored in the storage tank 12. By controlling the valve 27 to open the conveying pipe 26, the marsh gas will flow into the conveying pipe 26 from the storage tank 12, and then the marsh gas will be ejected by the at least one nozzle group 283 of the spray pipe 28 intermittently. Because of the relative positions of the two nozzles 2831 of the nozzle group 283 on the branches 282, the marsh gas will cause multiple vortexes in the organic substance 50 during the ejection to stir the organic substances 50. By the continuous and intermittent ejection, the present invention is able to increase the efficiency of the anaerobic digestion and make the anaerobic bacteria distributed evenly.

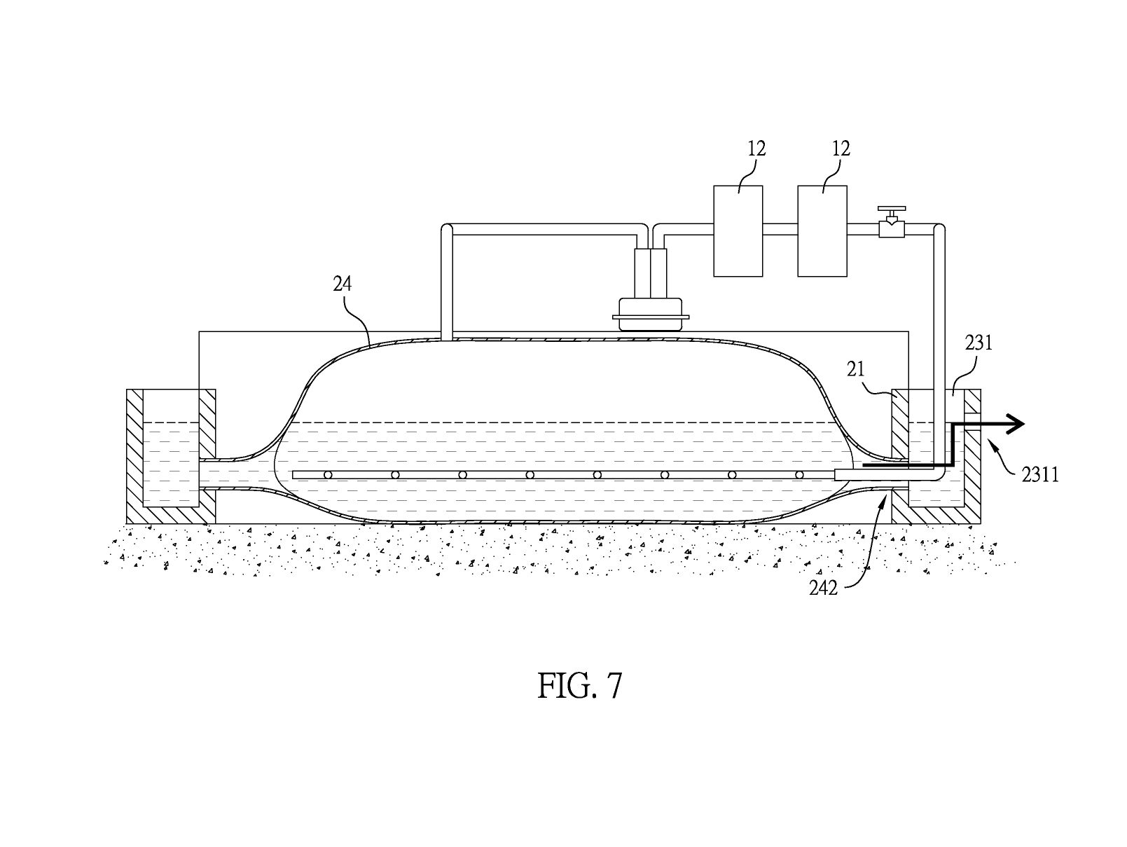

With reference to FIG. 7, after the anaerobic bacteria are fermented completely, the marsh gas that has been generated will be stored in the multiple storage tanks 12 for use as renewable energies. The marsh gas slurry will be drained by the outlet 2311 through the second opening 242 of the container 24 and the second gateway 231 of the second transporting base 23. The marsh gas slurry will be gathered and used in artificial wetlands, and the remaining organic fertilizer will be used for agriculture in order to promote the sustainable development for the environment.

By the above-mentioned technical feature, the present invention can use the circulation unit 10 and the spray pipe 28 to deliver the marsh gas that is generated during the fermentation, and make the marsh gas ejected by the nozzle groups 283 in order to stir the organic substances 50. By stirring the cycling marsh gas, the present invention is able to increase the efficiency of the anaerobic digestion and make the anaerobic bacteria distributed evenly.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. An anaerobic fermentation apparatus comprising:
a circulation unit having a pump and at least one storage tank communicating with each other; and
at least one fermentation tank, each one of the at least one fermentation tank having
a container having at least one opening;
a pumping pipe communicating with a top of the container and the pump;
a conveying pipe communicating with the at least one storage tank, mounted in one of the at least one opening of the container, and extending in the container;
a valve mounted on the conveying pipe and located outside the container; and
a spray pipe mounted in the container and having
a trunk communicating with an end of the conveying pipe and extending in the container; and
multiple branches being round tubes; each one of the multiple branches communicating with the trunk and having
at least one nozzle group; each one of the at least one nozzle group having two nozzles; the two nozzles formed through a tube wall of the branch; and an imaginary connection line that connects the two nozzles being oblique with respect to an axis of the branch as viewed from the top.

2. The anaerobic fermentation apparatus as claimed in claim 1, wherein in each one of the at least one nozzle group, two projections of the two nozzles on a section of the branch connect to the axis of the branch to form two imaginary connecting lines respectively; and an angle between the two imaginary connecting lines is from 10 to 15 degrees.

3. The anaerobic fermentation apparatus as claimed in claim 1, wherein in each one of the at least one nozzle group, a distance between the two nozzles along the axis of the branch is from 0.5 to 2 centimeters.

4. The anaerobic fermentation apparatus as claimed in claim 2, wherein in each one of the at least one nozzle group, a distance between the two nozzles along the axis of the branch is from 0.5 to 2 centimeters.

5. The anaerobic fermentation apparatus as claimed in claim 1, wherein the at least one nozzle group comprises multiple nozzle groups, and a distance between each two adjacent ones of the nozzle groups is from 20 to 40 centimeters.

6. The anaerobic fermentation apparatus as claimed in claim 4, wherein the at least one nozzle group comprises multiple nozzle groups, and a distance between each two adjacent ones of the nozzle groups is from 20 to 40 centimeters.

7. The anaerobic fermentation apparatus as claimed in claim 1, wherein the branches and the trunk are disposed on a same surface.

8. The anaerobic fermentation apparatus as claimed in claim 6, wherein the branches and the trunk are disposed on a same surface.

9. The anaerobic fermentation apparatus as claimed in claim 7, wherein the branches are divided into two groups respectively on two sides of the trunk; the two groups of the branches are staggered with respect to each other; the branches in the same group are spaced apart from each other.

10. The anaerobic fermentation apparatus as claimed in claim 8, wherein the branches are divided into two groups respectively on two sides of the trunk; the two groups of the branches are staggered with respect to each other; the branches in the same group are spaced apart from each other.

11. The anaerobic fermentation apparatus as claimed in claim 7, wherein the branches are divided into two groups respectively on two sides of the trunk; the two groups of the branches correspond in position; the branches in the same group are spaced apart from each other.

12. The anaerobic fermentation apparatus as claimed in claim 8, wherein the branches are divided into two groups respectively on two sides of the trunk; the two groups of the branches correspond in position; the branches in the same group are spaced apart from each other.

13. The anaerobic fermentation apparatus as claimed in claim 1, wherein each one of the at least one fermentation tank further comprises
a containment barrier surrounding a receiving space to accommodate the container;
a first transporting base mounted on an outer wall of the containment barrier and having
a first gateway formed in the first transporting base;
a first connecting opening formed through a wall of the containment barrier and communicating with the first gateway and the receiving space; and
a second transporting base mounted on the outer wall of the containment barrier opposite to the first transporting base and having
a second gateway formed in the second transporting base;
a second connecting opening formed through the wall of the containment barrier opposite to the first connecting opening and communicating with the second gateway and the receiving space.

14. The anaerobic fermentation apparatus as claimed in claim 12, wherein each one of the at least one fermentation tank further comprises
a containment barrier surrounding a receiving space to accommodate the container;
a first transporting base mounted on an outer wall of the containment barrier and having
a first gateway formed in the first transporting base;
a first connecting opening formed through a wall of the containment barrier and communicating with the first gateway and the receiving space; and
a second transporting base mounted on the outer wall of the containment barrier opposite to the first transporting base and having
a second gateway formed in the second transporting base;
a second connecting opening formed through the wall of the containment barrier opposite to the first connecting opening and communicating with the second gateway and the receiving space.

15. The anaerobic fermentation apparatus as claimed in claim 13, wherein the container has
a first opening formed through the container, facing the first transporting base, and communicating with the first gateway of the first transporting base via the first connecting opening; and
a second opening formed through the container, facing the second transporting base, and communicating with the second gateway of the second transporting base via the second connecting opening.

16. The anaerobic fermentation apparatus as claimed in claim 14, wherein the container has
a first opening formed through the container, facing the first transporting base, and communicating with the first gateway of the first transporting base via the first connecting opening; and
a second opening formed through the container, facing the second transporting base, and communicating with the second gateway of the second transporting base via the second connecting opening.

17. The anaerobic fermentation apparatus as claimed in claim 13, wherein the second transporting base has
an outlet formed through the second transporting base and communicating with the second gateway.

18. The anaerobic fermentation apparatus as claimed in claim 16, wherein the second transporting base has
an outlet formed through the second transporting base and communicating with the second gateway.

19. The anaerobic fermentation apparatus as claimed in claim 1, wherein the container is made of cement or brick and has
a first opening formed through a wall of the container;
a second opening formed through the wall of the container opposite to the first opening; and
a cover hermetically mounted on tops of inner walls of the container and forming an inner space; the inner space communicating with the first opening and the second opening; the pumping pipe formed through the cover and communicating with the inner space; the conveying pipe mounted in the second opening of the container and extending in the inner space of the container.

20. The anaerobic fermentation apparatus as claimed in claim 12, wherein the container is made of cement or brick and has
a first opening formed through a wall of the container;
a second opening formed through the wall of the container opposite to the first opening; and
a cover hermetically mounted on tops of inner walls of the container and forming an inner space; the inner space communicating with the first opening and the second opening; the pumping pipe formed through the cover and communicating with the inner space; the conveying pipe mounted in the second opening of the container and extending in the inner space of the container.
